(19) United States
(12) Patent Application Publication
(10) Pub. No.: US 2020/0383703 A1
(43) Pub. Date: Dec. 10, 2020

(54) INFLATABLE SYSTEM FOR CERVICAL DILATION AND LABOR INDUCTION HAVING SENSORS FOR MEASURING UTERINE INTERNAL PRESSURE AND FETAL HEART RATE

(71) Applicant: OB Tools Ltd., Nesher (IL)

(72) Inventors: Jack Atad, Haifa (IL); Tamir Ben-David, Tel Aviv (IL); Ilan Calderon, Bet Lechem Haglilit (IL); Arie Amara, Acco (IL); Yehuda Sharf, Tel Aviv (IL)

(73) Assignee: OB Tools Ltd., Nesher (IL)

(21) Appl. No.: 16/433,044

(22) Filed: Jun. 6, 2019

(51) Int. Cl. A61B 17/42; A61B 5/00; A61M 29/02

(52) U.S. Cl. CPC A61B 17/4241; A61B 5/4356; A61B 2017/00557; A61M 29/02; A61B 5/4362

(57) ABSTRACT

An inflatable system for cervical dilation and labor induction includes a uterine balloon, for positioning at a proximal portion of the uterus, adjacent to the cervical internal os, the uterine balloon being shaped to maximize the pressure against the decidua and the internal cervical os and to minimize the pressure on the fetal head. The inflatable system may have a vaginal balloon, for positioning in the vagina, for applying pressure on the external cervical os. The inflatable system may measure the pressure changes inside the balloon. The inflatable system may have electrodes for measuring the electrical activity of the fetal heart, the maternal heart and the uterine pressure activity and or uterine contractility and or labor progress. The inflatable system may measure cervical dilation, fetal well-being, and the woman's conditions.

### FIELD OF THE INVENTION

The present invention relates to a system and method of labor induction, and in particular, to a special intracorporeal system, comprising balloons, designed to induce cervical ripening, cervical dilation and labor induction, while performing monitoring tasks related to the fetal heart rate, (FHR), maternal heart rate (MHR), uterine contractility (UC) and other monitoring signals in labor.

### BACKGROUND OF THE INVENTION

There are various reasons to induce labor. For example, when a woman is two or more weeks overdue (postdate), and labor does not start on its own, it may be desirous to induce labor, due to fetal or maternal indications, such as placental dysfunction, pregnancy induced hypertension, preeclampsia, diabetes, intra-uterine growth restriction, or conditions that may jeopardize fetal well-being, or for the sake of fetal and maternal benefits of labor induction at gestational week 39 compared to expectant management (arrive trial, supported by Washington University) or other conditions that may affect the woman's health.

FIGS. 1A and 1B schematically illustrate woman's reproductive systems under normal conditions and during pregnancy.

FIG. 1A illustrates a woman's reproductive system 10, under normal conditions, showing a vagina 12, a cervix 16, forming a cervical canal 14, an external cervical os 15, an internal cervical os 17, a uterus 18, and an endometrium 21, being the mucous membrane lining the uterus 18.

Additionally, FIG. 1B illustrates a woman's reproductive system 20, during pregnancy, illustrating an amniotic sac 25 containing a fetus 27 having a head 29. A decidua 23 is the mucous membrane lining the uterus 18 in preparation for, and during, pregnancy.

Labor may be induced by causing the cervix 16 to soften and open. For example, the pharmaceutical substance, prostaglandin (PG) including misoprostol (Cytotec), leads to local biochemical and biophysical alterations in the cervical region that reduce cervical resistance and induce myometrial contractions.

A mechanical device for labor induction is also known. U.S. Pat. No. 4,976,692 to Atad describes a double-balloon catheter 30, illustrated in FIGS. 2A and 2B, designed to be placed in the woman's reproductive system 20 for about 12 hours or longer as needed to monitor uterine contractions and fetal heart rate activity, and/or fetal wellbeing as it is needed, until cervical dilatation progresses and achieves a dilatation diameter greater than the diameter of the inflated balloons, and/or spontaneous expulsion of the device occurs. The catheter 30 includes proximal and distal balloons 44 and 42, with respect to an operator, inflatable via lumens 34 and 38. When inserted, the balloon 42 is designed for placement in the uterus 18, for pressing against the internal cervical os 17, and the balloon 44 is designed for placement in the vagina 12, against the external cervical os 15. A section 46 of the catheter 30 is located between the balloons 44 and 42, in the cervical canal 14.

The catheter 30 may be used to introduce a medication via a third lumen 36. The proximal and distal balloons 44 and 42, are used to induce labor, by pressing against the openings of the external cervical os 15 and the internal cervical os 17, triggering hormone secretion from the decidua adjacent the internal cervical os 17, leading to cervical dilation and labor induction.

Another cervical dilator is known from US Patent Application 20040116955, which has balloon pressure measurement means. However, the pressure measurement has nothing to do with uterine contraction sensing, or fetal or maternal monitoring.

### SUMMARY OF THE INVENTION

The present invention successfully addresses the shortcomings of the presently known configurations by providing an inflatable system, of between one and three balloons, for cervical dilation and labor induction. The inflatable system may have a uterine balloon, for positioning at a proximal portion of the uterus, with respect to an operator, adjacent to the cervical internal os, the uterine balloon being shaped to maximize the pressure against the decidua and the internal cervical os and to minimize the pressure on the fetal head. Additionally or alternatively, the inflatable system may have a vaginal balloon, for positioning in the vagina, for applying pressure on the external cervical os. Additionally, the inflatable system may have sensors for measuring the pressure changes inside the uterine balloon. Additionally or alternatively, the inflatable system may have sensors for measuring the pressure and pressure changes inside the vaginal balloon.

The change in pressure in the uterine balloon is indicative of the uterine pressure build-up during uterine activity. Such pressure measurements can be used to measure uterine contractions, thereby taking the place of similar/equivalent pressure measurements done by using an intrauterine pressure catheter (IUPC). In addition, short-term changes in pressure inside the balloon (for example, in the range of 0.2-2 seconds) may correspond to fetal and maternal heart rates. Thus, the system of the invention can measure pressure and the pressure information can be used to extract fetal and maternal heart rates.

Additionally or alternatively, the inflatable system may have one or more inner uterine electrodes for measuring electrical activity of one or more of the following signals: the fetal heart activity, the maternal heart activity, the uterine electrical activity. Additionally or alternatively, the inflatable system may have cervical neck and/or vaginal electrode or electrodes for measuring electrical activity of one or more of the following signals: the fetal heart activity, the maternal heart activity, the uterine electrical activity. Additionally or alternatively, the inflatable system can be used together with external abdomen electrode or electrodes for measuring electrical activity of one or more of the following signals: the fetal heart activity, the maternal heart activity, the uterine electrical activity.

In some embodiments, the inner electrodes can be made from soft conducting material such as conductive silicone.

Additionally or alternatively, the inflatable system can be used together with additional sensors, such as imaging sensors, e.g., internal camera or ultrasound/echo sensors array for image beam forming and Doppler blood flow representation. These sensors can be placed in several locations such as the distal side of the uterine balloon, inside at least one of the balloons, adjacent to the cervical neck, from the uterine side and/or the vaginal side. In some applications, the imaging sensor can be used to measure and evaluate the cervical neck opening. In a specific embodiment, a camera or ultrasound probe is placed at the vaginal balloon, facing toward the cervical neck and used for measuring cervical neck opening.

Additionally or alternatively, the inflatable system can be used together with additional position sensors, such as an internal accelerometer or magnetic position sensor to track the catheter tip position and movements. These sensors can be placed in several locations such as the distal side of the uterine balloon, inside at least one of the balloons, adjacent to the cervical neck, from the uterine side and/or the vaginal side.

Additionally or alternatively, the inflatable system can be used together with an additional sound sensor, such as an internal microphone to detect the fetal and maternal heart beat sounds for FHR and MHR detection. These sensors can be placed in several locations such as the distal side of the uterine balloon, inside at least one of the balloons, adjacent to the cervical neck, from the uterine side and/or the vaginal side.

The above sensors and electrodes may be used with the inflatable system to monitor cervical dilation, fetal well-being, and the woman's conditions.

In some embodiments the cervical ripening and dilation is done both by balloons and by dedicated drugs, which are used for labor induction. In such cases, the sensors can be used to detect the effectiveness of balloon and/or drug treatment and optimize the required treatment. In specific cases, if the sensors detect an event of tachisystola, this may indicate that there is no need for using labor induction drugs.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-1B are schematic diagrams of a woman's reproduction system;

FIGS. 2A-2B schematically illustrate an inflatable system for cervical dilation and labor induction, from the prior art U.S. Pat. No. 4,976,692;

FIG. 3A is a simplified illustration of an inflatable system for cervical dilation, having a pressure measurement unit for measuring pressure changes inside a uterine balloon, in accordance with an embodiment of the invention;

FIG. 3B is a simplified illustration of an inflatable system for cervical dilation, having a pressure sensing probe for measuring pressure changes inside the uterine balloon, in accordance with an embodiment of the invention;

FIG. 3C is a simplified illustration of an inflatable system for cervical dilation, having a pressure sensor placed at the distal side of the uterine balloon, in accordance with an embodiment of the invention;

FIG. 4 is a simplified illustration of an inflatable system for cervical dilation, having a pressure measurement unit for measuring pressure changes inside the uterine balloon and the vaginal balloon, in accordance with an embodiment of the invention, simultaneously measuring pressure in both balloons;

FIG. 5 is a simplified illustration of an inflatable system for cervical dilation, having an electrode placed at the tip of the uterine balloon, in accordance with an embodiment of the invention;

FIGS. 6A and 6B are simplified illustrations of an inflatable system for cervical dilation, having an electrode placed at the tip of the uterine balloon (FIG. 6A) and an additional external abdomen electrodes (FIG. 6B), in accordance with an embodiment of the invention;

FIG. 6C is a simplified illustration of an external EMG electrode that cooperates with an inner uterine electrode of the system;

FIG. 6D is a simplified illustration of an external EMG electrode array that cooperates with an inner uterine electrode of the system;

FIG. 7 is a simplified illustration of an inflatable system for cervical dilation, having an electrode placed at the tip of the uterine balloon and an additional electrode placed at the cervical neck, in accordance with an embodiment of the invention;

FIG. 8 is a simplified illustration of an inflatable system for cervical dilation, having a bipolar electrode configuration placed at the tip of the uterine balloon, in accordance with an embodiment of the invention;

FIG. 9 is a simplified illustration of an inflatable system for cervical dilation, having an external expandable electrode jacket placed at the distal side of the uterine balloon, in accordance with an embodiment of the invention;

FIG. 10 is a simplified illustration of an inflatable system for cervical dilation, with one or more EMG electrodes adjacent the cervical neck between the proximal and distal balloons, simultaneously measuring EMG signals from electrodes on both balloons;

FIG. 11 is a simplified illustration of the system with a sensor at a distal face of the distal balloon; and

FIG. 12 is a simplified illustration of the system with an inner uterine EMG electrode or sensor that can be disconnected from the balloon device.

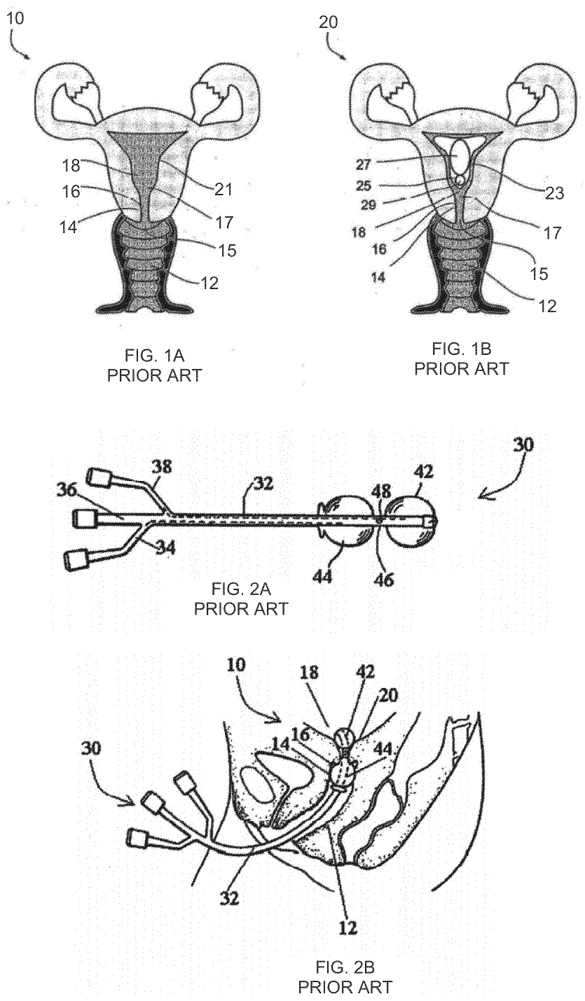

### DETAILED DESCRIPTION

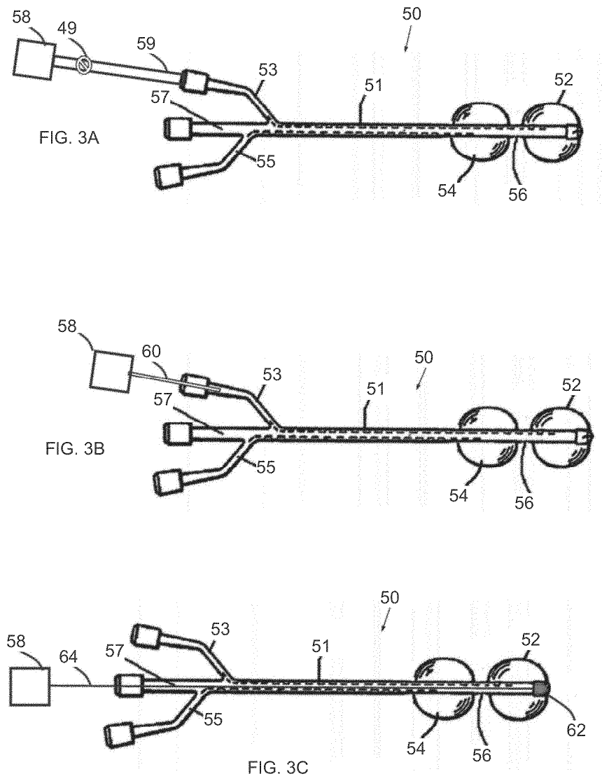

Reference is now made to FIG. 3A, which illustrates an inflatable system 50 for cervical dilation, having pressure measurement unit for measuring pressure changes inside the uterine balloon, in accordance with an embodiment of the invention.

Inflatable system 50 may include distal and proximal balloons 52 and 54, which are inflatable via first and second lumens 53 and 55, respectively, formed in a catheter 51. When inserted in the patient, the distal balloon 52 may be placed in the uterus for pressing against the internal cervical os. Proximal balloon 54 may be placed in the vagina against the external cervical os. An intermediate portion 56 of catheter 51, between distal and proximal balloons 52 and 54 may be positioned in the cervical canal.

Catheter 51 may include a third lumen 57, which may be used to introduce a medication or other substances or tools. The proximal and distal balloons 54 and 52 may be used to induce labor, by pressing against the openings of the external cervical os and the internal cervical os, thereby triggering hormone secretion from the decidua adjacent to the internal cervical os, leading to cervical dilation and labor induction.

A pressure measuring unit 58 may be in communication with distal balloon 52 via first lumen 53 and an optional fluid connector 59. The pressure measuring unit 58 may be any suitable unit (e.g., mechanical, electronic, pneumatic, hydraulic, etc.) for measuring the pressure in distal balloon 52. An optional fluid valve 49 may be provided, such as between pressure measuring unit 58 and the coupling to first lumen 53, for regulating the fluid connection between pressure measuring unit 58 and distal balloon 52.

FIG. 3B illustrates another version of inflatable system 50. In this version, the pressure measuring unit 58 is coupled to first lumen 53 via a pressure sensing probe (tube) 60.

FIG. 3C illustrates another version of inflatable system 50. In this version, the pressure measuring unit 58 is electrically coupled to a pressure sensor 62 via a wire 64, which may be positioned through third lumen 57 or any other lumen. For example, pressure sensor 62 may be a piezoelectric pressure sensor, capacitance pressure sensor, resistive pressure sensor and the like. The pressure sensor 62 may be mounted at the distal face of distal balloon 52 (either on the external or internal surface of the balloon).

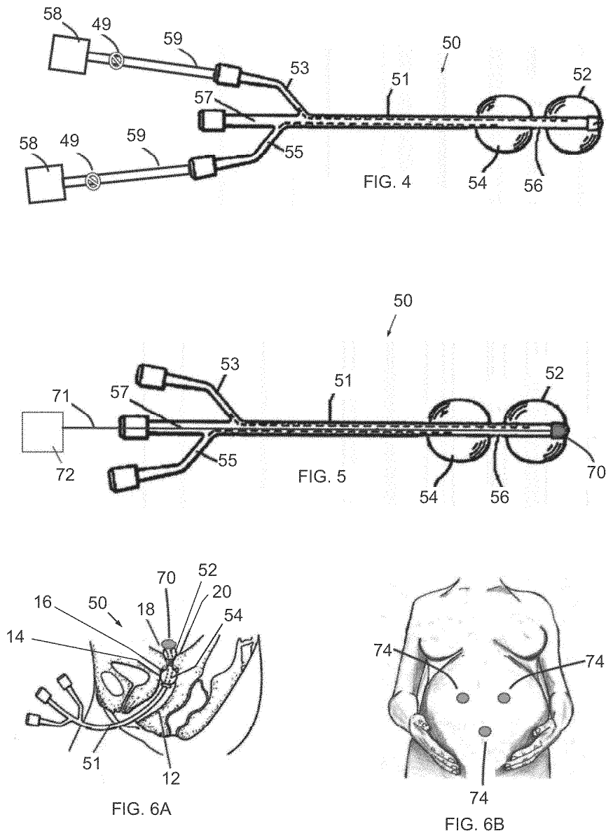

FIG. 4 illustrates another version of inflatable system 50. In this version, one pressure measuring unit 58 is coupled to distal balloon 52 and another pressure measuring unit 58 is coupled to proximal balloon 54.

Reference is now made to FIG. 5, which illustrates another version of inflatable system 50 for cervical dilation. In this version, system 50 includes a uterine electromyocardiogram (EMG) electrode 70 mounted at the distal face of distal balloon 52 (either on the external or internal surface of the balloon). EMG electrode 70 is in electrical communication with an EMG unit 72, such as by wired connection with a wire 71, or by wireless communication. EMG unit 72 includes electronics for energizing electrode 70 and processing components for processing the electrical signal from electrode 70, as is known in the art. EMG electrode 70, as processed by EMG unit 72, can sense electrical uterine activity and/or fetal heartbeat.

The inflatable system 50 of FIG. 5 may include the pressure measurement elements of any of FIGS. 3A-4; optionally, the inflatable system 50 of FIG. 5 may be used without the pressure measurement elements.

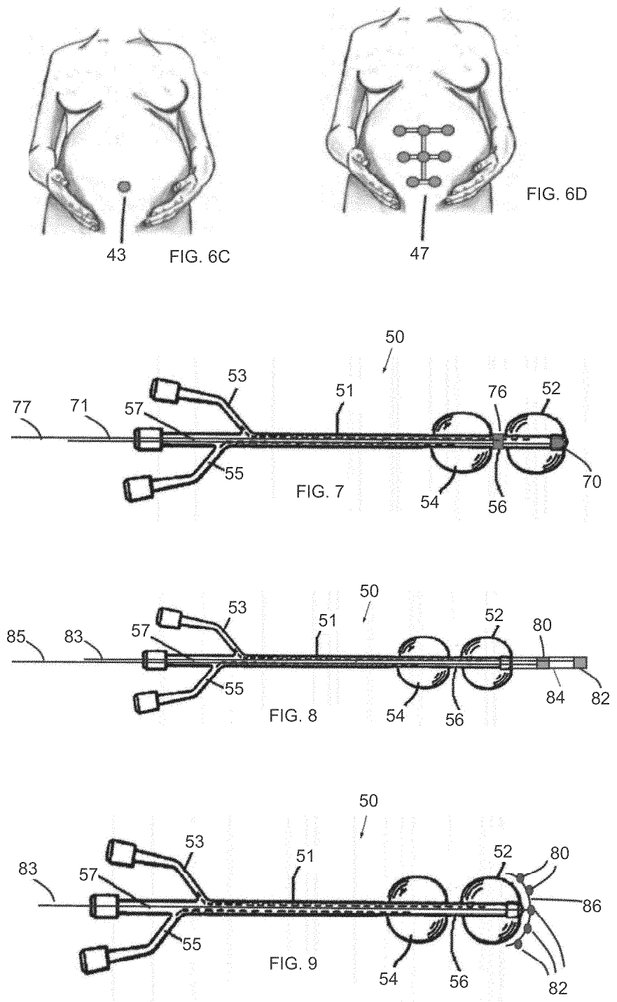

Reference is now made to FIGS. 6A and 6B. The inflatable system 50 is placed in the patient with EMG electrode 70 placed at the tip of the uterine balloon 52. The electrode 70 may be operated in a bipolar mode in conjunction with one or more additional external abdomen electrodes 74, as seen in FIG. 6B, or an external EMG electrode 43 (FIG. 6C) or an external EMG electrode array 47 (FIG. 6D).

Reference is now made to FIG. 7, which illustrates another version of the inflatable system 50 of FIG. 5. In this version, an additional electrode 76 is placed at the cervical neck portion 56 and may communicate wirelessly or by a wire 77 with the EMG unit 72 (not shown here).

Reference is now made to FIG. 8, which illustrates another version of the inflatable system 50 of FIG. 5. In this version, first and second electrodes 80 and 82, operating in bipolar mode, are distal to distal balloon 52 and placed in a sleeve 84 coupled distally to catheter 51. The first and second electrodes 80 and 82 may communicate wirelessly or by wires 83 and 85 with the EMG unit 72 (not shown here).

Reference is now made to FIG. 9, which illustrates another version of the inflatable system 50 of FIG. 5. In this version, first and second electrodes 80 and 82 (there may be more than one electrode 82, as shown), operating in bipolar mode, are distal to distal balloon 52 and placed in an external expandable electrodes jacket 86 coupled distally to catheter 51.

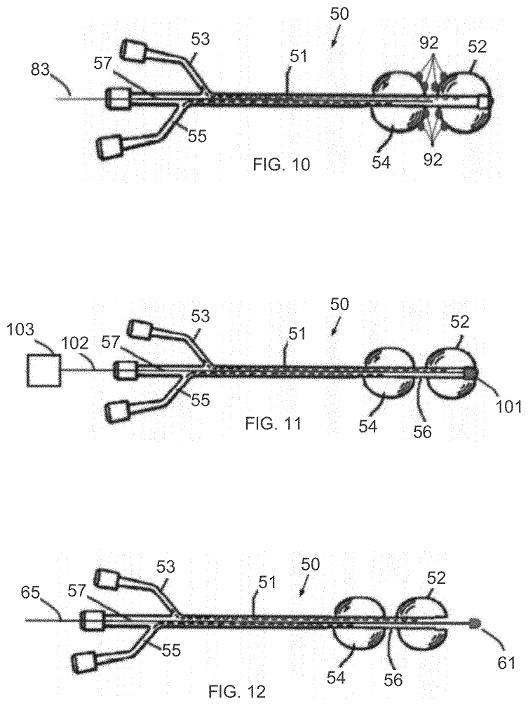

Reference is now made to FIG. 10, which illustrates another version of the inflatable system 50 of FIG. 5. In this version, one or more EMG electrodes 92 are adjacent the cervical neck between the proximal and distal balloons 54 and 52. Electrodes may be placed at other advantageous positions as well.

Reference is now made to FIG. 11, which illustrates another version of the inflatable system 50 of FIG. 5. In this version, an imaging sensor 101 is coupled to a distal portion of the distal balloon 52 (e.g., inside or outside the balloon). The imaging sensor 101 may be coupled to an image processing unit 103 wirelessly or by a wire 102. The imaging sensor 101 may be a camera, ultrasonic probe, Doppler blood flow sensor, or any other imaging modality sensor. Imaging sensor 101 can be placed at several locations, such as without limitation, the distal side of the uterine balloon, inside at least one of the balloons, adjacent to the cervical neck, from the uterine side and/or the vaginal side. In some applications, the imaging sensor can be used to measure and evaluate the cervical neck opening. In one example, a camera or ultrasound probe is placed at the vaginal balloon, facing the cervical neck, and can image and measure the cervical neck opening.

Reference is now made to FIG. 12, which illustrates another version of the inflatable system 50 of FIG. 5. In this version, an inner uterine EMG electrode or sensor 61 is coupled to a guide element 65 that extends distally beyond distal balloon 52. In this manner, the electrode or sensor is disconnected from the balloon device and can be manipulated independently of the balloon device.

What is claimed is:

1. An inflatable system comprising:
a labor induction balloon mounted on a catheter formed with one or more lumens for inflation of said balloon;
a sensor coupled to said balloon, said sensor comprising at least one of a fetal heart rate sensor, a maternal heart rate sensor and a uterine activity sensor; and
a processor coupled to said balloon and said sensor, said processor being configured to selectively control operation of said balloon to perform a labor induction action or to process information from said sensor to measure at least one of a fetal heart rate, a maternal heart rate and a uterine activity.

2. The inflatable system according to claim 1, wherein said sensor is located inside said balloon.

3. The inflatable system according to claim 1, wherein said sensor is located in a lumen coupled to said balloon.

4. The inflatable system according to claim 1, wherein said sensor is in fluid contact with said balloon.

5. The inflatable system according to claim 1, wherein said sensor comprises a pressure sensor operative to sense pressure changes in said balloon.

6. The inflatable system according to claim 1, wherein said sensor comprises a uterine electromyocardiogram (EMG) electrode.

7. The inflatable system according to claim 6, further comprising one or more additional external abdomen electrodes that cooperate with said EMG electrode.

8. The inflatable system according to claim 1, wherein said sensor comprises an imaging sensor.

9. The inflatable system according to claim 1, wherein said sensor comprises a position sensor.

10. The inflatable system according to claim 1, wherein said sensor comprises an acoustic sensor.

11. The inflatable system according to claim 1, wherein said balloon comprises a proximal and a distal balloon mounted on said catheter, and an electrode is coupled to said catheter between said proximal balloon and said distal balloon.

12. The inflatable system according to claim 1, wherein said sensor comprises at least two electrodes distal to said balloon.

13. The inflatable system according to claim 12, wherein said at least two electrodes are placed in a sleeve coupled distally to said catheter.

14. The inflatable system according to claim 12, wherein said at least two electrodes are placed in an external expandable electrodes jacket coupled distally to said catheter.

15. The inflatable system according to claim 1, wherein said sensor is coupled to a guide element that extends distally beyond said balloon.

16. A method comprising using the system of claim 1 to control operation of said balloon to perform a labor induction action in a pregnant woman or to process information from said sensor to measure at least one of a fetal heart rate, a maternal heart rate and a uterine activity.

17. The method according to claim 16, wherein said balloon is positioned at a uterus of the woman near an internal cervical os of the woman, and wherein said balloon is shaped and inflated to increase pressure against a decidua and the internal cervical os of the woman and to lower pressure on a fetal head of a fetus in the woman, and further comprising measuring internal pressure of said balloon and correlating said pressure to uterine activity.

18. The method according to claim 16, wherein said balloon is positioned at a vagina of the woman adjacent to an external cervical os of the woman, and wherein said balloon is shaped and inflated to increase pressure against the external cervical os.

19. The method according to claim 16, wherein said sensor comprises an electrode and further comprising using said electrode to measure electrical activity of at least one of the fetal heart rate, the maternal heart rate and uterine activity.
